(19) United States
(12) Patent Application Publication
(10) Pub. No.: US 2014/0378334 A1
(43) Pub. Date: Dec. 25, 2014

(54) METHOD FOR QUANTIFYING RENAL MARKERS BY ASSAYING URINE

(71) Applicant: ASSISTANCE PUBLIQUE - HOPITAUX DE PARIS (APHP)-, PARIS CEDEX (FR)

(72) Inventors: Pierre Galichon, Paris (FR); Eric Rondeau, Vincennes (FR); Isabelle Brocheriou, Paris (FR); Yi-Chun Xu-Dubois, Paris (FR); Alexandre Hertig, Paris (FR)

(21) Appl. No.: 14/353,262

(22) PCT Filed: Oct. 19, 2012
(86) PCT No.: PCT/EP2012/070821

§ 371 Date: Apr. 21, 2014

(30) Foreign Application Priority Data
Oct. 20, 2011 (FR) 1159485

(51) Int. Cl. C12Q 1/68

(52) U.S. Cl. CPC C12Q 1/6883; C12Q 2600/158
USPC 506/9; 435/6.12; 506/16; 435/6.11

(57) ABSTRACT

The invention pertains to a method of in vitro diagnosis of pathologies in a patient. According to the invention, the method comprises the following steps:
a) obtaining a urine sample from said patient,
b) detecting, in said sample, at least one marker of said pathology and at least one specific marker of the urothelial cells and/or the urothelial microparticles, and
c) determining a threshold of expression of said at least one marker of said pathology by normalization of said marker of said pathology by said at least one specific marker of the urothelial cells and/or the urothelial microparticles.

### 1. FIELD OF THE INVENTION

The field of the invention is that of uronephrology. More specifically, the invention pertains to a method of in vitro diagnosis of pathologies of the urinary system.

### 2. PRIOR ART

Kidney diseases can affect the different structural compartments of the kidney: the vessels, the glomeruli, the tubules or the interstitium. These disorders lead to acute and/or chronic kidney failure, their ultimate development being the total destruction of the functional units of the kidney which are replaced by an expansion of the extra-cellular matrix, i.e. renal fibrosis. These kidney diseases have various corresponding etiologies: obstruction of the excretory tracts, inflammation, auto-immunity, allergy, deposition diseases, hypertension, diabetes, vasculopathies, ischemia, toxicity, etc. Kidney diseases can affect native kidneys or allotransplants after a kidney transplant. In France, it is estimated that there are 3,000 new cases of transplants (kidney, heart, liver, bone marrow, lungs, etc) every year. The systematic follow-up of patients having received transplants has enabled the study of the early stages of renal diseases evolving towards fibrosis. The expression in the renal tissue of epithelial-mesenchymal transition (EMT) markers enables early detection of a fibrosing disease in the renal tissues, which can be caused by ischemia, the rejection or toxicity of immunosuppressants, especially cyclosporin A (CsA) (Slattery et al, Am J Pathol. 2005 August; 167(2): 395-407; Hertig et al, American Journal of Transplantation 2006, Galichon et al, Fibrogenesis Tissue Repair 2011; Galichon et al, Transplantation, 2011).

EMT is a dynamic process during which the cells lose their epithelial characteristics and acquire mesenchymal characteristics. These modifications affect the morphology of the cell as well as its working. When EMT reaches the renal tubular cells, it progresses towards fibrosis and chronic renal failure (Hertig et al, J Am Soc Nephrol 2008). It is therefore necessary to monitor the appearance of this phenomenon among patients who have undergone transplants in order to adapt or modify the immunosuppressant treatment.

At present, the reference method implemented for the detection and monitoring of any renal pathology is biopsy. Biopsy consists in removing a core of tissue from the kidney by transcutaneous, transvenous or surgical means. This sample is then subjected to a histological examination to detect possible signs of pathology (destruction, cell infiltration or hypertrophy of the glomerular, tubular, vascular or interstitial compartments).

This method however has numerous drawbacks. Taking samples is not without risks for the patient. Many complications have been observed such as hematuria, obstructive renal failure and even anuria, hematoma in the perirenal region, the appearance of arterial and venous fistulas and more rarely hemorrhage, loss of transplant, and death. Apart from the risks related to any invasive procedure, it can happen that the biopsies are performed in a region that does not represent the overall condition of the kidney and that, therefore, the patient's true situation is under-estimated or over-estimated because of this sampling procedure.

In addition, since biopsies cannot be done systematically at short intervals, this method does not enable early detection of the appearance of a pathology either. Besides, performing a renal biopsy is costly, complex, invasive and painful for the patient.

It is therefore necessary to find a non-invasive, simple, economical, reliable method of diagnosis that enables early detection and entails the least possible risk for the patient.

### 3. GOALS OF THE INVENTION

The invention is aimed at overcoming these drawbacks of the prior art.

More specifically, it is a goal of the invention, in at least one embodiment, to provide a method of early diagnosis of renal pathologies or of pathologies having renal repercussions.

It is another goal of the invention, in at least one embodiment, to implement a method of non-invasive diagnosis.

It is yet another goal of the invention, in at least one embodiment, to implement a reliable and precise method of diagnosis.

It is another goal of the invention, in at least one embodiment of the invention, to implement a method of diagnosis that is simple to perform.

It is another goal of the invention, in at least one embodiment, to implement a more economical method of diagnosis.

It is another goal of the invention, in at least one embodiment, to implement a method for following up the efficacy and tolerance of a treatment.

Finally, it is another goal of the invention, in at least one embodiment, to implement a method of diagnosis that is less costly for the patient.

### 4. SUMMARY OF THE INVENTION

These goals as well as others that shall appear here below are achieved entirely or at least partly by means of a method of in vitro diagnosis of pathologies in a patient.

According to the invention, such a method comprises the following steps:
a) obtaining a urine sample from said patient,
b) detecting, in said sample, at least one marker of said pathology and at least one specific marker of the urothelial cells and/or the urothelial microparticles, and
c) determining a threshold of expression of said at least one marker of said pathology by normalization of said marker of said pathology by said at least one specific marker of the urothelial cells and/or the urothelial microparticles.

Thus, the invention relies on the use of cells and microparticles contained in the urine in order to extract therefrom the genetic material and to compare the expression of a gene of interest, correlated with a pathology, with the expression of a specific gene of the urine cells unaffected by the pathology.

Urine indeed contains a small quantity of urothelial cells arising out of the normal renewal of the epithelium of the urinary excretory tracts. It can also contain quantities, variable depending especially on the presence of a renal pathology, of leukocytes, renal tubular or glomerular cells, blood as well as microparticles.

The term “microparticles” is understood to mean complex vesicular structures that can be released by most cells during the activation process or apoptosis. They are formed by a bilayer membrane of phospholipids exposing transmembrane proteins and receptors, and they enclose cytosolic constituents such as enzymes, transcription factors and mRNA coming from their mother cells.

The term “urothelial cells” is understood to mean transitional epithelial cells forming the human urothelium, from the pelvis up to the urethra. These cells have various shapes: cylindrical, kite-shaped, umbrella-shaped and balloon-shaped.

The term “specific marker of urothelial cells” is understood to mean a gene specifically expressed by the urothelial cells or urothelial microparticles, whether it is within the cells or on the surface, the level of synthesis of this gene by said cell being independent of the pathologies that can effect the renal cells. This notion is therefore different from the notion of a housekeeping gene, the expression of which is ubiquitous whatever the cell type, the function of the cell or its state.

Although this is rare, it is possible using current-day methods of molecular biology or biochemistry to extract nucleic acids and proteins from the cell and the microparticles contained in the patient's urine. In order to eliminate the bias related to the quantity of cells and microparticles in the sample, it is common practice to express the expression of the gene of interest as a function of the expression of a housekeeping gene such as GAPDH (glyceraldehyde 3 phosphate dehydrogenase) genes, 18S ribosomal RNA, the cyclophilin A or B, the β-catenin or again HPRT (hypoxanthine-guanine phosphoribosyltransferase) (Absolute quantification of mRNA using real-time reverse transcription polymerase chain reaction, S. A. Bustin, Journal of Molecular Endocrinology, 2000, 25, 169-193).

However, this method does not take account of the cellular specificity of the pathology. Thus, the modification of the expression of a gene linked to a pathology affecting a precise cell type can be masked by the fact that the housekeeping gene coming from other cell types present in the urine is strongly expressed.

One of the contributions of the invention is therefore that it normalizes the expression of the gene of interest by the expression of a gene independent of the cell types affected. This step of normalization is used to determine a threshold of expression of the pathological marker by means of a urine marker independent of the quantitative and qualitative variations of the urine cells of renal origin. It is then possible to know whether this marker is expressed strongly or, on the contrary, weakly. This characteristic makes it possible to obtain a diagnostic test that is reliable, precise and reflects the patient's state of health with greater exactness. Thus, the method according to the invention can be used for diagnostic purposes to monitor the progress of a pathology.

In addition, working from a urine sample has many advantages:
the sample is easy to access;
collecting the sample is non-invasive and painless;
collecting the sample is economical.

The simplicity of this method also makes it possible to implement it in all types of laboratories without making use of special technical qualifications or equipment other than that commonly used. The absence of any particular investment for implementing this method thus reduces the costs of analysis.

Finally, this method of normalization can be applied to different pathologies, renal or non-renal, provided that these pathologies modify the expression and/or quantity of urothelial cells and/or microparticles excreted and present in the urine.

The invention furthermore pertains to a method of in vitro diagnosis in which the step b) comprises the detection of the product of transcription of said at least one specific marker of the urothelial cells and/or urothelial microparticles and of the product of transcription of said at least one marker of said pathology.

The study of the products of transcription is more reliable than the study of the presence of a gene in the genome of the cell. It is indeed well known that the presence of a gene in the genome of a cell cannot necessarily be correlated with its expression in said cell, since the regulation of the expression of a particular gene is subject to numerous parameters. The detection of the product of transcription therefore makes it possible to obtain a more precise and more reliable result.

Another object of the invention is a method in which the step b) is implemented by means of a technique of amplification of nucleic acids chosen from the group comprising RT-PCR, quantitative PCR, final-point PCR, semi-quantitative PCR or their combination.

The term PCR (Polymerase Chain Reaction) is understood to mean the technique in which a fragment of target DNA is replicated in vitro in numerous copies. The term RT-PCR (Reverse Transcriptase Polymerase Chain Reaction) is understood to mean in vitro synthesis of a complementary DNA from extracted messenger RNAs. The term “quantitative PCR”, also known as real-time PCR, is understood to mean the technique of in vitro replication of a fragment of target DNA additionally enabling measurement of the initial quantity of this target fragment. Semi-quantitative PCR can be distinguished from quantitative PCR in that the PCR is interrupted at several points enabling the initial quantity of DNA to be evaluated. This type of PCR is useful when the quantity of DNA is unusually low. Final-point PCR associates the Northern Blot technique with classic PCR in order to evaluate the initial quantity of DNA by comparison of the bands on agarose gel.

These methods, which cost little to implement, have the advantage of giving a fast and reliable result compatible with the requirements of a diagnostic test.

The invention furthermore pertains to a method of in vitro diagnosis in which the step b) is implemented using a nucleic acid hybridization technique chosen from the group comprising in situ hybridization (ISH), fluorescence in situ hybridization (FISH) or hybridization with marking by fluorescence (FISH), biochip hybridization, the Northern Blot method or the Southern Blot method.

The invention also pertains to a method of in vitro diagnosis in which the step b) is implemented through a method of sequencing of the nucleic acids.

Yet another object of the invention is a method of in vitro diagnosis in which the pathology is a renal pathology chosen from the group comprising renal fibrosis, a phenotypic change of the renal epithelial cells, a transplant rejection, a cancer, the glomerular diseases (diabetes, extramembranous glomerulonephritis, minimal glomerular lesions, segmentary and focal hyalinosis, etc), the tubular diseases (acute tubular necrosis, expression of epithelial-to-mesenchymal transition markers, atrophy, cellular rejection, obstruction of the excretory tracts, etc), the interstitial diseases (inflammation, fibrosis) and the vascular kidney diseases (arterial hypertension, thrombotic microangiopathy, humoral rejection, etc).

The term “epithelial-mesenchymal transition” (EMT) refers to a biological process that enables a polarized epithelial cell, interacting normally with the basal membrane, to undertake numerous biochemical transformations that enable it to acquire a mesenchymal cell phenotype, including increased migratory capacity, an invasive character, increased resistance to apoptosis and massive increase in the components of the extra-cellular matrix (Kalluri R, Weinberg R A, “The basics of epithelial-to-mesenchymal transition”, J Clin Invest. 119 (2009) 1420-1428). The epithelial phenotypic changes are the EMT markers (for example vimentin and R-catenin in the tubular epithelium) that can be studied in the tissues in a clinical situation (Hertig A. et al.n “Early epithelial phenotypic changes predict graft fibrosis”, J Am Soc Nephrol. 19 (2008) 1584-1591).

Another object of the invention is a method during which said patient has received an organ transplant and said renal pathology is the presence of an interstitial fibrosis, a tubular atrophy or epithelial-mesenchymal transition in the renal transplant.

The method according to the invention therefore enables the efficient and early detection of the emergence of a renal pathology such as inflammation or EMT-inducing epithelial phenotypic changes in the kidney.

Yet another object of the invention is a method of in vitro diagnosis in which at least one specific genetic marker of said renal pathology is chosen from the group comprising the human genes CD45 (SEQ ID 1), CD68 (SEQ ID 2), and VIM (SEQ ID 3) as well as the genes having a sequence homology of at least 80%, preferably at least 85%, preferably at least 90%, preferably at least 95%, preferably at least 99% with these human genes.

By way of a precise indication, the human gene CD45 is also symbolized as PTPRC (Protein Tyrosine Phosphatase Receptor type C). In the following description, the human gene CD45 or PTRPC will be designated equally by the symbol CD45 or PTPRC.

The inventors have surprisingly discovered that the expression of these genes is considerably increased in the urine of patients having undergone clinically stable kidney transplants but for which, however, the biopsy of the transplant reveals the presence of epithelial phenotypic changes. This over-expression can be correlated with the presence of epithelial phenotypic changes arising during an epithelial-mesenchymal transition and tubular-interstitial diseases in the biopsies of renal allotransplants, these biopsies being performed in the context of a systematic screening three months after the transplant. It is possible, as understood in the invention, to search for the expression of only one gene, which is a specific marker of a pathology. However, in order to refine the diagnosis, it is preferable to search for a combination of different genes, the expression of which in urine takes account of the presence of a particular renal pathology. For example, we can cite research of the expression of CD45, or PTPRC, and CD68 genes, normalized by uroplakin to detect the presence of inflammatory cells in the transplant. It is also possible to search for the expression of certain tumor markers. Examples that can be cited are markers for clear-cell carcinoma, the search for racemase, caveolin-1 (SEQ ID 29), ROR1 (SEQ ID 30), CD10 (SEQ ID 31), keratin 7, vimentin (SEQ ID 3), TP53 (SEQ ID 26) in the context of monitoring a renal cancer.

A “homologous sequence” or a “sequence homology” between nucleotide sequences is determined by linear comparison of the nucleotide sequences using the software BLAST (Basic Local Alignment Search Tool), using the algorithm blastn available on the NCBI site: (http://blast.ncbi.nlm.nih.gov/Blast.cgi?PROGRAM=blastn&BLAST_PROGRAMS=megaBlast&PAGE_TYPE=BlastSearch&SHOW_DEFAULTS=on&LINK_LOC=blasthome).

The parameters chosen for this analysis are the following:
database: human genomic plus transcript (Human G+T);
no exclusion of models or samples of environmental sequences;
optimizing of the program: blastn (somewhat similar sequences);
short queries=automatically adjusted parameters for input sequence;
expect threshold=10;
word size=11;
max match in a query range=0.

With respect to the scoring parameters, these parameters are fixed by default (match/mismatch scores=2-3; gap costs=existence: 5, extension: 2). Finally, no filter is applied.

According to another advantageous embodiment, said pathology is a pathology modifying the quantity of cells and/or microparticles excreted in the urine.

Preferably, said pathology modifying the quantity of cells and/or microparticles excreted in the urine is chosen from the group comprising glomerular diseases such as segmentary and focal hyalinosis, tubular diseases such as acute tubular necrosis, epithelial phenotypic changes, cell rejection and interstitial diseases such as acute transplant rejection, Sjögren's syndrome and sarcoidosis.

Thus, the method according to the invention enables the reliable, speedy and non-invasive detection of the development of non-renal diseases through the collection of urine samples from the patient, when the pathologies modify the profile of gene expression and/or the quantity of cells excreted in the urine.

Tubular necrosis takes the form of an increase in the number of tubular cells in the urine due to a major desquamation of the walls of the renal tubular epithelium. Segmentary or focal hyalinosis is accompanied by a major quantity of podocytes in the urine. The increase in the number of leukocytes is a sign of acute rejection of a transplant.

Advantageously, said at least one specific marker of urothelial cells is chosen from the group comprising the human genes uroplakin 1A (SEQ ID 4), uroplakin 1B (SEQ ID 5), uroplakin 2 (SEQ ID 6), uroplakin 3A (SEQ ID 7), uroplakin 3B (SEQ ID 8), uroplakin 3BL (SEQ ID 9), Bcas1 (SEQ ID 10), CEP152 (SEQ ID 11), CRABP2 (SEQ ID 12), DNASE1 (SEQ ID 13), KRT20 (SEQ ID 14), PLEKHF1 (SEQ ID 15), PLEKHG4B (SEQ ID 16), RCN1 (SEQ ID 17), SEMA5B (SEQ ID 18), SULT2A1 (SEQ ID 19), TFF1 (SEQ ID 20), VILL (SEQ ID 21), ZNF720 (SEQ ID 22) as well as genes having sequence homology of at least 80%, preferably at least 85%, preferably at least 90%, preferably at least 95%, preferably at least 99% with these genes.

Among the cells present in the urine, only the urothelial cells express these genes. These genes are specifically and constantly expressed by the urothelial cells and/or microparticles, independently of the renal pathologies. Naturally, these genes are present in the genetic material contained in the nucleus of each cell of the organism. However, the genes are not expressed in the same way by all the cells of the organism. In other words, not all the genes are transcribed from DNA to mRNA and then translated from mRNA into protein in all the cells forming the human body. However, the inventors have discovered that, among the cells and microparticles contained in urine, these genes are specifically expressed by the urothelial cells and that that they are so expressed constantly. They therefore constitute referentials of choice for normalizing genes linked to a pathology specifically affecting the renal cells or modifying their quantity in the urine. The inventors wish to emphasize that the above-mentioned genes can in fact be detected in other cell types, for example when this detection is based on a simple search for the presence of a gene in the total DNA and not in the genes expressed by a cell type. These genes can also be expressed by other types of cells. However, their interest in the present invention is related to the fact that, among all the cell types that can be found is a patient's urine sample, only the urothelial cells express these genes, independently of pathological conditions. Consequently, as understood in the invention, the notion of a specific marker of the urothelial cells or of the urothelial microparticles must be distinguished from the notion of a housekeepinghousekeeping gene. Indeed, housekeepinghousekeeping genes are genes expressed by all the cells whatever their cell type and their function. On the contrary, the term “specific marker of the urothelial cells” corresponds to genes expressed solely by the urothelial cells among all the cells that can be found in a urine sample.

Besides, these genes have been identified by the inventor as genes that can be used to obtain an excellent statistical correlation (p value<0.01) relative to this method of normalization by the housekeeping genes (18S RNA, GAPDH, etc). The p value obtained for each gene is indicated in Table 1 below.

TABLE 1
Specific markers of the urothelial cells

| Name of the gene | p-value |
|---|---|
| UPK1A | 1.37 · 10⁻⁴⁰ |
| UPK1B | 1.40 · 10⁻¹² |
| UPK2 | 2.11 · 10⁻¹² |
| UPK3A | 4.04 · 10⁻⁹ |
| Bcas1 | 4 · 10⁻⁵ |
| PLEKHF1 | 2.17 · 10⁻⁵ |
| KRT20 | 3.05 · 10⁻⁵ |
| ZNF720 | 7.80 · 10⁻⁵ |
| UPK3B | 1.74 · 10⁻⁴ |
| RCN1 | 1.97 · 10⁻⁴ |
| TFF1 | 2.54 · 10⁻⁴ |

Preferably, said at least one specific marker of the urothelial cells or of the urothelial microparticles is chosen from the group of genes comprising the human genes UPK1A, UPK1B, UPK2 and UPK3A as well as the genes having a sequence homology of at least 80%, preferably at least 85%, preferably at least 90%, preferably at least 95%, preferably at least 99% with these genes.

The term “normalization” is understood to mean the elimination of biases related to errors of measurement or manipulation and independent of the biological variations. The step of normalization therefore enables the production of more reliable results. Normalizing by using specific genes of the cells affected by the pathology being sought eliminates the bias related to the proportion of these same cells in the urine sample. Indeed, the quantitative and qualitative composition of urine is not fixed either because of the rate of flow of urine or because of the disease. The risk of wrongly estimating the true progress of the pathology in the patient is therefore real and could prove to be detrimental to his therapeutic treatment.

For example, when the quantitative PCR technique was used, the normalization consisted of a passage from the logarithmic scale (corresponding to the raw result) to a linear scale in two steps:

Cp_{specific marker of urothelial cells}−Cp_{pathological marker}=ΔCₚ, (1)

the Cp, or crossing point, being the number of cycles of amplification before detection of the fluorescent signal by the apparatus.

Level of expression of the normalized pathological marker=2^{ΔCp} (2)

When a migration on gel is implemented to display results as is the case for the classic PCR followed by migration on agarose gel or for the Northern and Southern Blot techniques, the operator must measure the optical densities of each migration band with any usual software (Image J™, UN-SCAN-IT Gel™) and make a report of it to determine the threshold of expression of the gene of interest in the patient. The threshold of expression is then computed as follows:

Level of expression of the normalized pathological marker=optical density of pathological marker/optical density of urothelial cell marker

Another object of the invention is a method of in vitro diagnosis further comprising a step for comparing the threshold of expression of said marker of a renal pathology with a threshold of expression unchanged by the disease.

The comparison with a healthy patient makes it possible to note the positive or negative influence of a pathology on the regulation of the expression of a normally expressed gene. It is done as follows:

Regulation of a gene=2^{(ΔCp patient-ΔCp healthy individual)}

Apart from the comparison with a healthy patient, it is possible to monitor the progress of a transplant or a pathological state through the method of the invention. It is indeed possible to keep the previous results of a patient and to use them as a referential. This internal normalization eliminates the bias due to the differences between individuals. It also enables the medical follow-up of the patient and the monitoring of his illness or of the transplant.

The invention further comprises an in vitro diagnostic kit for the detection of pathologies from a urine sample coming from a patient, the kit comprising at least one pair of primers for the detection, in said sample, of at least one specific marker of a pathology and at least one pair of primers for the detection, in said sample, of at least one specific marker of the urothelial cells.

Advantageously, said pathology is a renal fibrosis or a phenotypic change of the renal epithelial cells, and said at least one specific marker of a renal pathology is chosen from the group comprising the human genes CD45, CD68 and VIM.

In a preferred embodiment, said at least one specific marker of urothelial cells is chosen from the group comprising the human genes uroplakin 1A, uroplakin 1B, uroplakin 2, uroplakin 3A, uroplakin 3B, uroplakin 3BL, Bcas1, CEP152, CRABP2, DNASE1, KRT20, PLEKHF1, PLEKHG4B, RCN1, SEMA5B, SULT2A1, TFF1, VILL, ZNF720 as well as genes having a sequence homology of at least at least 80%, preferably at least 85%, preferably at least 90%, preferably at least 95%, preferably at least 99% with these human genes.

Another object of the invention lies in the use of the in vitro diagnostic kit for the detection of a renal pathology, said renal pathology being a fibrosis or a phenotypic change of the renal epithelial cells.

### 5. LIST OF FIGURES

Other features and advantages of the invention shall appear more clearly from the following description of a preferred embodiment, given by way of a simple illustratory and non-exhaustive example and from the appended drawings, of which:

FIG. 1 is a graph showing the correlation of the EMT scores with the result of the urine PCR for vimentin (VIM) normalized by GAPDH.

FIG. 2 illustrates the correlation of the same EMT scores with the same results of urine PCR for vimentin (VIM) when the results are normalized by the uroplakin 1A gene UPK1A.

FIG. 3 is a graph representing the correlation of the EMT scores with the results of the urine PCR for CD68 normalized by GAPDH.

FIG. 4 shows the correlation of the same EMT scores with the same results of urine PCR for CD68 when these results are normalized by the uroplakin 1A gene UPK1A.

FIG. 5 represents the correlation of the EMT scores with the results of the urine PCR of CD45 normalized by GAPDH.

FIG. 6 represents the correlation of the same EMT scores with the same results of urine PCR for CD45 when these results are normalized by the uroplakin 1A gene UPK1A.

FIG. 7 is a graph representing the number of identified genes corresponding to the terms “kidney” and “the inter-cell junction” according to the method of the invention.

FIG. 8 is a graph representing the significance of gene enrichment with respect to the term “kidney” and the term “inter-cell junction” according to the method of normalization.

### 6. DESCRIPTION OF ONE EMBODIMENT OF THE INVENTION

The general principle of the invention relies on the comparison of the expression of a gene correlated with a pathological phenomenon, designated as a marker of a pathology or pathological marker, with the expression of a reference gene, the level of expression of which in the urothelial cells is independent of the cells affected by the pathology. This marker is designated as a specific marker of the urothelial cells.

### EXAMPLE 1

### Diagnosis of the Epithelial Phenotypic Changes of the Renal Transplant through the Method of the Invention

In order to evaluate the sensitivity of the diagnostic method according to the invention, renal biopsies are carried out on patients who have undergone an organ transplant and have been treated with CsA. At the same time, a sample of their urine is collected. In these samples, a search is made by quantitative PCR for markers associated with a phenotypic change of the renal epithelial cells. In order to demonstrate the superiority of the method according to the invention, the results of quantitative PCR are normalized according to a urothelial reference gene, in compliance with the method according to the invention, and according to a housekeepinghousekeeping gene, in compliance with the classic method described in the literature.

Control biopsies on transplant patients are analyzed by the Anatomopathology Laboratory of the Hôpital Tenon (Paris). A search is made for the protein expression of vimentin and β-catenin, which are EMT markers, according to methods of immunohistochemistry well known to those skilled in the art. The EMT score is determined as a function of the percentage of renal tubules expressing the EMT markers, i.e. vimentin and β-catenin (Hertig et al, American Journal of Transplantation 2006). These scores are expressed as follows:
score=0: no EMT;
score=1: <10% of the renal tubules in the biopsy present EMT markers;
score=2: 10-24% of the renal tubules in the biopsy present EMT markers;
score=3: 25-50% of the renal tubules in the biopsy present EMT markers;
score=4: >50% of the renal tubules in the biopsy present EMT markers.

The patient is considered to be positive for EMT when the score is greater than or equal to 2.

1. Collection of Urine and Preparation of Cellular Lysate

50 ml of fresh urine is collected from these patients in a Falcon® tube. The collection is done during the three weeks preceding the biopsy in order to prevent the presence of red blood cells in the urine. The selected patients have no trace of urinary infection, and have not had any residual diuresis before the transplant. The day's first miction is not used.

The urine sample is centrifuged at 2000 rpm for 20 minutes, at ambient or room temperature (T_{amb}). A volume of 2 ml of supernatant is stored at −80° C. The rest of the supernatant is discarded. The cell pellet containing cells and minerals is taken into a volume of 15 ml of buffer solution PBS1×. The cell suspension is again centrifuged to remove debris for 10 minutes at 2000 rpm, at T_{amb}. The supernatant is discarded, the pellet is drained by the overturning of the tube and then re-suspended in 150 μl of lysis buffer RLT, supplemented with 1% by volume of β-mercaptoethanol (14.3 M solution). The buffer RLT is provided by the Qiagen laboratories in the RNeasy® Micro Kit. At this step, the lysate thus obtained can be kept at −80° C. or directly used to extract RNA.

2. Extraction of RNA

The RNA messengers (mRNA) are extracted from the cells present in the urine sample. The markers indicating pathology, fibrosis or EMT are generally expressed only when these phenomena appear. To study their transcription is therefore more relevant than to look for their presence in the genome.

The RNA is extracted from the cellular lysate, prepared as described here above, using the RNeasy® Micro Kit (Qiagen) according to the protocol provided by the manufacturer. More specifically, the protocol followed is the protocol “Tissues obtained by micro-dissection”. Briefly, a volume of 70% sterile ethanol is added to the homogenized lysate according to the indications of the protocol. The lysate is deposited entirely or partly in a RNeasy® column provided with the kit. The columns are centrifuged for 15 seconds at a rotational speed of over 10,000 rpm at 4° C. The flow-through is discarded. The column is washed with buffer RW1 provided with the kit. The RNA is eluted and then recovered in 14 μl of water without RNAse.

3. Complementary DNA Synthesis

The reverse transcription of the RNA extracted here above is achieved by means of the QuantiTect® Reverse Transcription kit (Qiagen). Briefly, the RNA solution produced previously is added to the gDNA Wipeout Buffer provided with the kit and then incubated at 42° C., for 2 minutes. This step eliminates the residual genomic DNA. The reverse transcription mix (RT Primer Mix) contains nucleic bases, reverse transcriptase (Quantiscript® Reverse Transcriptase) and the reaction buffer (Quantiscript® RT Buffer). It is added to the RNA solution. The mixture is incubated for 15 minutes at 42° C., so that reverse transcription is achieved. The mixture is then incubated for 3 minutes at 95° C., in order to deactivate the reverse transcriptase. The solution of complementary DNA thus ready can be preserved or diluted to 1/10^{th} before analysis.

4. Quantitative PCR

Quantitative PCR is used to evaluate the initial quantity of transcription products in the cells. It therefore makes it possible to determine whether a gene is over-regulated or under-regulated.

Briefly, a reaction mixture is prepared containing:
5 μl of SYBR® Green Master Mix 2×(Roche Laboratories)/well,
0.25 μl of each primer at 10 μM (Roche Laboratories)/well,
1.5 μl of sterile water/well.
   7 μl of this reaction mixture is deposited per well in a 96-well plate (Roche Laboratories). 3 μl of complementary DNA from each patient, diluted to 1/10^{th}, is added to each well. The plate is then centrifuged at 1500 g for 2 minutes and then introduced into the LightCycler 480 automaton (Roche Laboratories) for amplification.

The pairs of primers used are provided by the Roche Laboratories:

TABLE 2
Sequences of pairs of primers of human genes
vimentin, CD45 (or PTPRC), GAPDH
and uroplakin 1A

| | Sense | Anti-sense |
|---|---|---|
| Gene | primer | primer |
| VIM | gaccagctaac | gaagcatctc |
| | caacgacaaa | ctcctgcaat |
| CD45 | agttattgttatgc | tgctttccttc |
| | tgacagaactgaa | tccccagta |
| CD68 | gtccacctcgac | cactggggcagg |
| | ctgctct | agaaact |
| Uroplakin | ggtagccagttt | agcatgagcac |
| 1A (UPK1A) | tggtgtgg | caggtacg |
| GAPDH | agccacatcgc | gcccaatacga |
| | tcagacac | ccaaatcc |

Vimentin is a protein belonging to the family of intermediate filaments. Its gene symbol is VIM. It takes part in the cytoskeleton. CD45 or PTPRC is a transmembrane protein tyrosine phosphatase normally expressed by the leukocytes. CD68 is a glycoprotein normally expressed by macrophages and monocytes.

The genes of vimentin (VIM), CD68 and CD45 (PTPRC), in this case our genes of interest, seem to be particularly over-expressed in the renal tubules and/or renal interstitium during fibrosing diseases of the transplant, which are manifested also in immunohistochemistry by the presence of EMT in renal biopsies. The GAPDH gene is used as a reference housekeeping gene, and is expressed in all types of nucleated cells without distinction. The uroplakin 1A gene is used as a reference gene specific to the urothelial cells.

The following is the amplification program:
1 pre-incubation cycle (5 minutes at 95° C.)
45 amplification cycles (15 seconds at 60° C.; 15 seconds at 72° C.)
1 fusion curve (5 seconds at 96° C., 1 minute at 60° C., then slow heating from 0.06° C./s to reach 96° C. with 10 acquisitions/° C.).
1 cooling cycle (30 seconds at 40° C.).

The plate containing amplified DNA is withdrawn from the automaton and then preserved at 4° C. The raw data are retrieved from the automaton for normalization.

5. Normalization

The raw data are normalized according to the reference method used to compute the initial quantity of DNA during a quantitative PCR. Briefly, the results of each patient were normalized and then linearized as follows:
relative to a housekeepinghousekeeping gene (GAPDH):

Cp_{GAPDH}−Cp_{gene of interest}=ΔCp,

Gene of interest/GAPDH=2 ^{ΔCp}
relative to uroplakin, which is the specific marker of the urothelial cells:

Cp_{UPK}−Cp_{gene of interest}=ΔCp,

Gene of interest/uroplakin=2^{ΔCp}

Cp being the number of amplification cycles before detection of the fluorescent signal by the apparatus.

6. Results

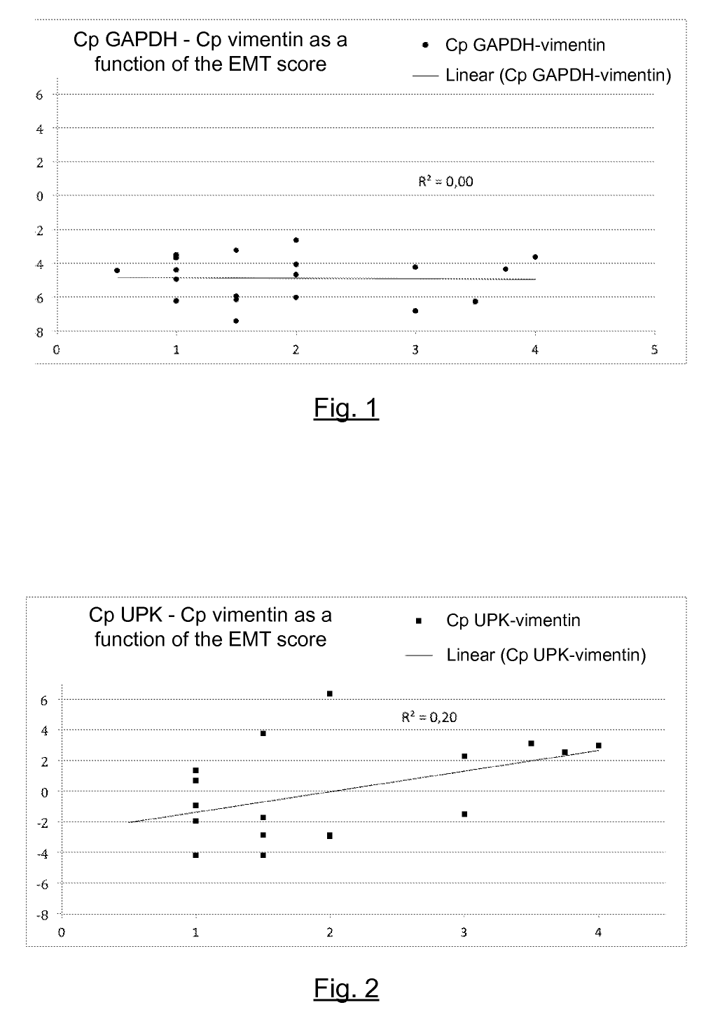

FIGS. 1 and 2 present the correlation of the results of the quantitative PCR on the vimentin gene in correlation with the EMT scores. In FIG. 1, the correlation coefficient R² is very low and the slope of the regression line is zero. It is therefore impossible to conclusively relate the expression of vimentin to the presence of epithelial phenotypic change in the renal cells. However, the normalization of the results by the uroplakin in compliance with the method of the invention significantly improves the regression coefficient. Furthermore, the slope of the regression line becomes positive, thus clearly and unequivocally correlating the expression of the vimentin gene with increasingly higher EMT scores. These results are therefore consistent with the results of biopsies.

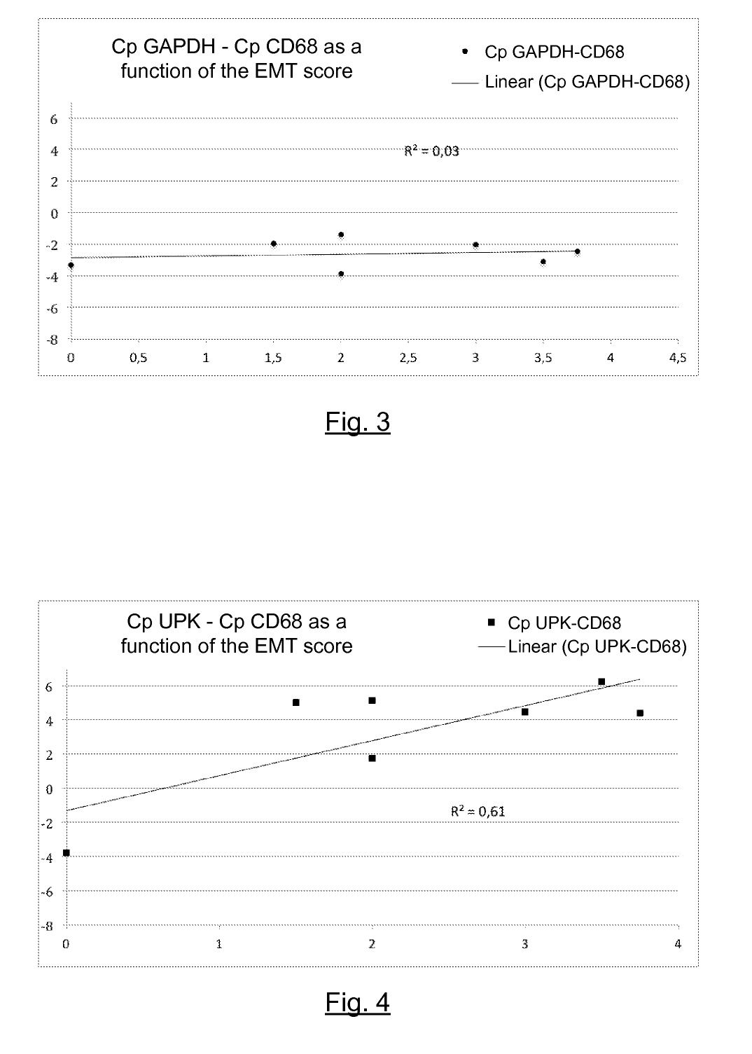

Similarly, according to FIG. 3, the slope of the regression line relating the expression of CD68 with the presence of EMT is very low. This would mean that the expression of CD68 is not correlated with the appearance of epithelial phenotypic changes in the kidney. Now, the expression of CD68 is positively regulated in renal fibrosing diseases (Anders et al, Kidney Int., 2011). It is therefore clear that the classic method of normalization by a housekeeping gene leads to false negatives.

On the contrary, FIG. 4 shows that normalization by uroplakin considerably improves the test. The slope of the regression straight line becomes positive and the expression of CD68 is positively regulated during the phenomena of epithelial phenotypic changes. The result is in accordance with the anatomopathological examination on the control biopsies.

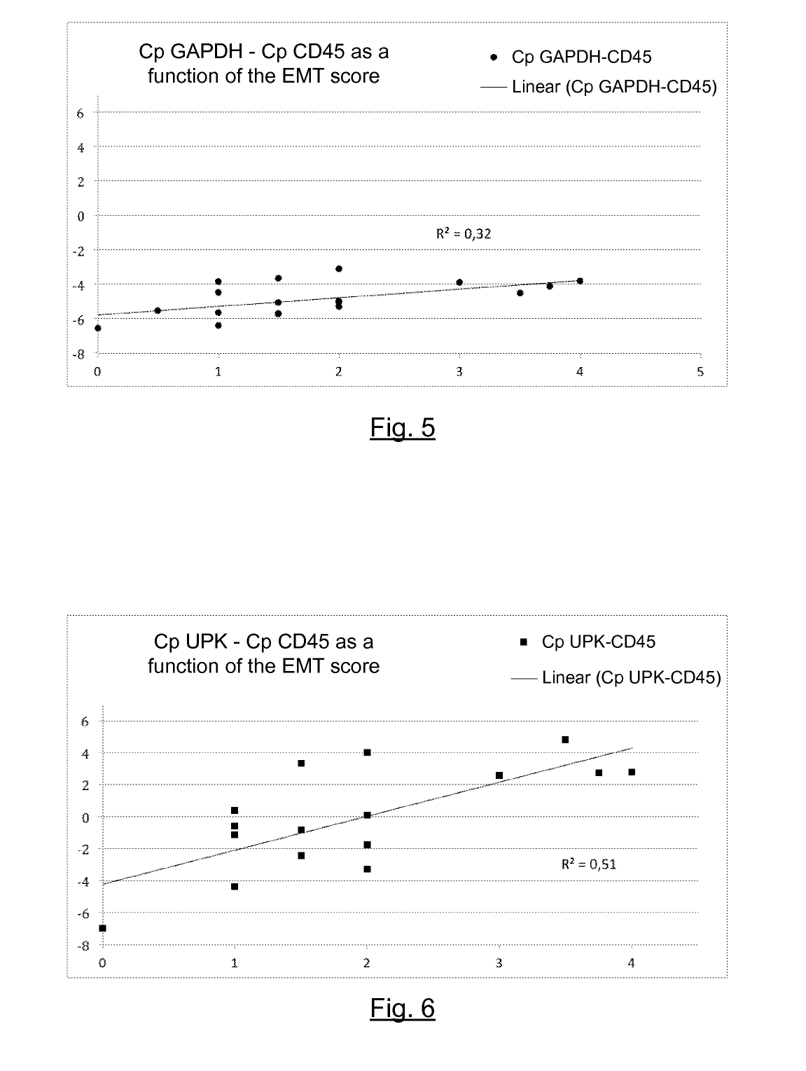

With respect to the expression of CD45 (PTPRC), the over-expression of CD45 (PTPRC) in the renal tissue is associated with an unfavorable development of the kidney allotransplants (Scherer et al, Nephrol Dial. Transplant., 2009). The comparison of FIGS. 5 and 6 shows that the PCR test using urine is considerably improved when the normalization is done by uroplakin.

In conclusion, the normalization of the results relative to GAPDH does not enable any efficient discrimination between patients showing EMT and “healthy” patients, and this is the case whatever the gene of interest studied. As indicated by the slopes, respectively zero and negative, of the regression lines of FIGS. 1 and 3, the normalization by the GAPDH housekeeping gene leads to false negatives. The clinical specialist therefore cannot rely on the results of this type of analysis. Resorting to a confirmation biopsy therefore remains inevitable.

The test is considerably improved when uroplakin is used as the reference gene for normalizing the results. According to FIGS. 2, 4 and 6, the expression of vimentin, CD68 and CD45 (PTPRC) is regulated positively in phenomena of EMT in the kidney. This corresponds to what has been effectively observed in immunohistochemistry in biopsies on patients. Thus, the method according to the invention reflects the patient's real situation. It also enables precise and reliable monitoring and diagnosis of the appearance of epithelial phenotypic changes in the kidney.

It is therefore clear that the method according to the invention gives results that are reliable, precise and consistent with the patient's real situation. Furthermore, this method is painless for the patient, swift, simple and economical to implement. It is furthermore perfectly suited to the monitoring of the appearance of EMT in a patient who has undergone an organ transplant.

### EXAMPLE 2

### Detection of Genes Expressed in the Urine of 26 Patients Showing or Not Showing Epithelial Phenotypic Changes in a Biopsy of a Transplant, and Comparison of the Results Obtained with the Classic Method of Normalization and the Method According to the Invention

26 clinically stable patients had urine samples taken as described in example 1 before biopsy of the transplant. Of the 26 patients analyzed, 12 showed no signs of EMT, while 14 showed signs of EMT. Cell pellets were prepared from these urine samples as described in Example 1. These cell pellets were then sent to the firm Miltenyi Biotech Gmbh for extraction of RNA, complementary DNA reverse transcription, amplification, or incorporation of the DNA fluorescent marker, quality controls and hybridization on complementary DNA microarrays from Agilent®. These microarrays are used to make a quantitative study of the expression of the genes representing the totality of the human genes. The transcriptome of each patient was therefore analyzed on a microarray. In other words, one microarray corresponds to one patient. The level of expression of the genes is expressed in intensity of fluorescence after adjustment on internal fluorescence references present in each microarray: these are raw data. The median corresponds here to the luminosity emitted and recorded in the gene situated on the median of the list of genes analyzed on the complementary DNA microarray.

Normalization by the median eliminates the bias related to the preparation of each of the microarrays. An example of bias related to the preparation of the microarray is the quantity of fluorescent marker incorporated in the patient's DNA or the temperature to which the microarray is exposed. Normalization by the median is done by applying the following formula to each gene tested on the complementary DNA microarray for the given patient:

Normalized value=(raw value)/(median of the values of all the genes tested on the microarray)

A generalized linear model of a binomial family was created by using the R software:

model←glm(EPC˜x, family=binomial, offset=blood+SFN)

where:
model is the model,
EPC is the predicted binary categorical variable (indicating the presence or non-presence of epithelial phenotypic changes in the biopsy),
x represents each gene tested on the complementary DNA microarray tested separately in this model,
the blood and SFN co-variables are respectively the mean value of the hemoglobin genes and the mean value of stratifin on the basis of measurements of expression delivered by the complementary DNA microarray of a given individual.

Taking blood and stratifin as co-variables makes it possible to takes account of the contamination of the collected sample by blood and/or non-renal epithelial cells.

The significance (p value) of the improvement of the prediction of the EMT variable by the introduction of x into the module is given by the following command:

anova(model,test=“Chisq”)[2.5].

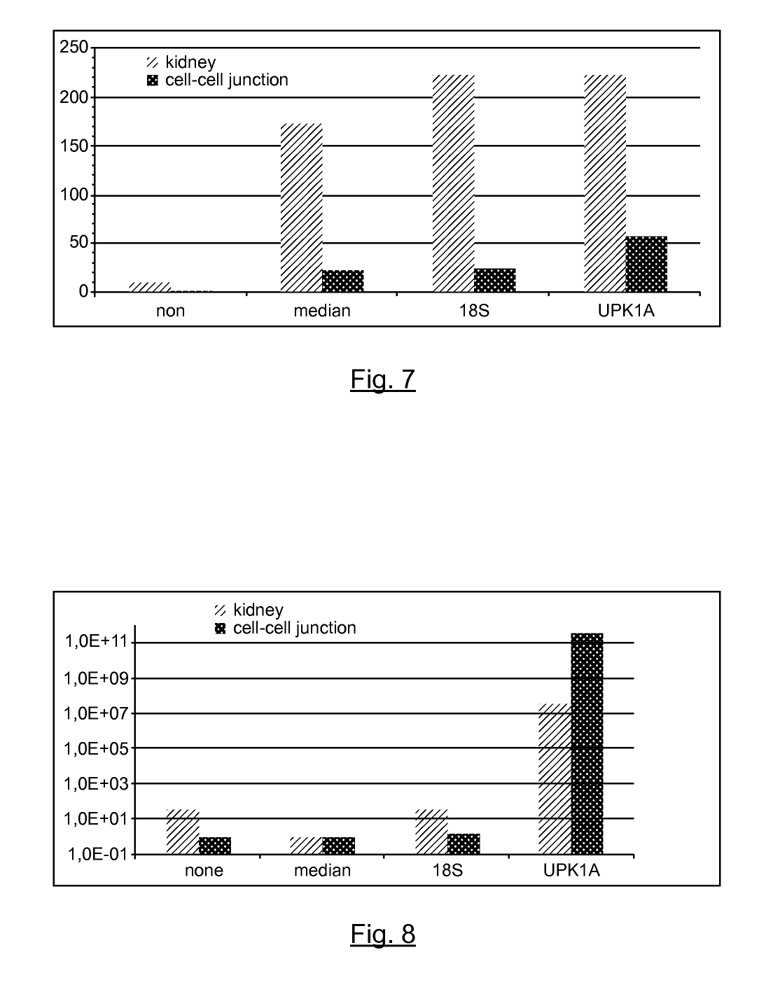

The genes, the obtained p value of which is <0.05, were selected for each method of normalization and used as a list of genes for the functional study of enrichment by means of the DAVID software (david.abcc.ncifcrf.gov). The totality of the genes assessed by the complementary DNA microarray was used as a reference list (background). These results are presented in FIGS. 7 and 8 as well as in Table 3 here below:

TABLE 3
Enrichment of the terms “kidney” and “intercell junction”
according to the normalization method

| | | | Number | | Value p ad- |
|---|---|---|---|---|---|
| Normal- | | | of genes | Enrichment | justed by the |
| ization | Data base | Term | identified | coefficient | Bonferroni method |
| None | UP_TISSUE | Kidney | 8 | 2.68 | 7.15E−01 |
| None | GOTERM_CC_FAT | GO:0005911~cell- | 1 | 2.31 | 1.00E+00 |
| | | cell junction | | | |
| Median | UP_TISSUE | Kidney | 172 | 0.90 | 1.00E+00 |
| Median | GOTERM_CC_FAT | GO:0005911~cell- | 22 | 0.80 | 1.00E+00 |
| | | cell junction | | | |
| 18S | UP_TISSUE | Kidney | 220 | 1.15 | 9.98E−01 |
| 18S | GOTERM_CC_FAT | GO:0005911~cell- | 24 | 1.01 | 1.00E+00 |
| | | cell junction | | | |
| UPK1A | UP_TISSUE | Kidney | 222 | 1.42 | 1.13E−05 |
| UPK1A | GOTERM_CC_FAT | GO:0005911~cell- | 57 | 2.70 | 1.39E−09 |
| | | cell junction | | | |

The inventors made observations firstly of the enrichment of the term “kidney” in the UP_TISSUE data base in order to evaluate the consistency of the results obtained by implementing the method of normalization according to the invention relative to the organ studied, and secondly of the enrichment of the “cell-cell junction” (GO :0005911˜cell-cell junction) in the GOTERM_CC_FAT data base in order to evaluate the consistency of the results relative to the pathology studied, in this case EMT.

Table 3 and the FIGS. 7 and 8 which are taken from Table 3 show that the normalization by uroplakin 1A is used to obtain the most significant enrichment for these two terms, an enrichment which remains significant solely for normalization by uroplakin 1A when correction by the Bonferroni method is applied to take account of multiple tests. Normalization by uroplakin 1A identifies the greatest number of genes belonging to these two terms as associated with the presence of epithelial phenotypical changes in the renal biopsy.

### 7. APPLICATIONS

The method according to the invention has thus demonstrated its efficiency in the early detection of the appearance of phenotypic changes. Other applications in the detection of EMT can be obtained by the method of the invention. The detection of acute rejection of a renal transplant can be diagnosed through the method according to the invention. In this case, the pathological markers sought will be the markers related to the activation of the immune cells in the kidney such as granzyme B (SEQ ID 23), perforin (SEQ ID 24), the interferons or Fas-Ligand (SEQ ID 25).

The detection of tubular, podocyte or inflammatory cells could be used, through the method according to the invention, for the diagnosis of any kidney disease. The progress of renal cancer in a patient could also be monitored through the method according to the invention through the detection of the markers TP53 (SEQ ID 26), MIB1 (SEQ ID 27), AgNOR, CD44 (SEQ ID 28), racemase, CD10 (SEQ ID 31), keratin 7, vimentin, caveolin-1 (SEQ D 29) and ror1 (SEQ ID 30).

1. Method of in vitro diagnosis of pathologies in a patient comprising the following steps:
a) obtaining a urine sample from said patient,
b) detecting, in said sample, at least one marker of said pathology and at least one specific marker of the urothelial cells and/or the urothelial microparticles, and
c) determining a threshold of expression of said at least one marker of said pathology by normalization of said marker of said pathology by said at least one specific marker of the urothelial cells and/or the urothelial microparticles.

2. Method according to claim 1 wherein the step b) comprises the detection of the product of transcription of said at least one specific marker of the urothelial cells and/or urothelial microparticles and of the product of transcription of said at least one marker of said pathology.

3. Method according to claim 1 wherein the step b) is implemented by means of a technique of amplification of nucleic acids chosen from the group comprising RT-PCR, quantitative PCR, final-point PCR, semi-quantitative PCR or their combination.

4. Method according to claim 1, wherein said pathology is a renal pathology chosen from the group comprising renal fibrosis, a phenotypic change of the renal epithelial cells, a transplant rejection, a cancer, the glomerular diseases, the tubular diseases, the interstitial diseases and the vascular kidney diseases.

5. Method according to claim 4, wherein said at least one specific genetic marker of said renal pathology is chosen from the group comprising the human genes CD45, CD68 and VIM as well as the genes having a sequence homology of at least 80%, preferably at least 85%, preferably at least 90%, preferably at least 95%, preferably at least 99% with these genes.

6. Method according to claim 1, wherein said pathology is a pathology modifying the quantity of cells and/or microparticles excreted in the urine.

7. Method according to claim 6, wherein said pathology modifying the quantity of cells and/or microparticles excreted in the urine is chosen from the group comprising segmentary and focal hyalinosis, acute tubular necrosis, epithelial phenotypic changes, acute transplant rejection, Sjögren's syndrome, sarcoidosis.

8. Method according to claim 1, wherein said at least one specific marker of urothelial cells and/or urothelial microparticles is chosen from the group comprising the human genes uroplakin 1A, uroplakin 1B, uroplakin 2, uroplakin 3A, uroplakin 3B, uroplakin 3BL, Bcas1, CEP152, CRABP2, DNASE1, KRT20, PLEKHF1, PLEKHG4B, RCN1, SEMA5B, SULT2A1, TFF1, VILL, ZNF720 as well as genes having a sequence homology of at least 80%, preferably at least 85%, preferably at least 90%, preferably at least 95%, preferably at least 99% with these genes.

9. Method according to claim 1, further comprising a step for comparing the threshold of expression of said marker of a normalized pathology with a threshold of gene expression, the expression of which is not modified by the disease.

10. In vitro diagnostic kit for the detection of a phenotypic change of the renal epithelial cells from a urine sample from a patient, comprising at least one pair of primers for the detection, in said sample, of at least one specific marker of a pathology chosen from the group comprising the human genes CD45, CD68 and VIM and at least one pair of primers for the detection of uroplakin 1A.

11. Use of the in vitro diagnostic kit according to claim 10 for the detection of a renal pathology, said renal pathology being epithelial phenotypic changes of the kidney.
